SEQUENCE LISTING (8 sequences)

SEQ ID NO: 1 - protein, 379 aa, Suberites domuncula
MSAILKRNVPIQRVGLPLTSYVSRWASALPTRTHPFYKLVDDSTTPVTRSTLLSAHMVDTLLDENQQSRHENQHTDTSYKMYQGLKFVVKTLFTPSKCHRHFSTSAHLSAMGRHQSPINIITSSTTKGPSLKPLKFSKSWDKPVIGTVKDTGYYLKFAPESAAEKCTLHTYNGEYILDHFHYHWGKKDGEGAEHFIDGKQYDIEFHFVHKKVGLTDPDARDAFAVLGVFGKADPRLKINGIWELLSPSTVLTVDSTRNVADVVPSKLLPSARDYFHYEGSLTTPTYGEVVHWFVLNEPIAVPSEYLSALRQMQADKEGTVIDSNYRELQEVHNRPVQRFKSDEQGRGEFDDISKNEDIVEDLSKLSGNFIRELVRKIYW

SEQ ID NO: 2 - DNA, 1396 bp, Suberites domuncula
gaattcggcacgagggacaactttgcataacttttactgtccatgtttaacgtttagatctagtactagtagtctacaagaacaactgtcaacaactgtcagattatgtgtataaaccaagatgtctgcaattcttaagagaaacgtacctatccaaagagtcggtctcccactgacctcctatgtcagtagatgggcttctgctctgcccaccaggacccatcctttttacaagttggttgatgacagtaccaccccagtgacaaggtctactcttctcagtgctcatatggttgacaccttgctagatgagaaccagcagagcagacatgaaaaccaacacacagacacgtcttacaaaatgtaccagggattaaaatttgttgtaaagacgctgtttactccatcgaaatgccaccgtcacttctccacatcagctcatttgtctgccatgggtcgacatcaatcccccatcaatataatcacctccagtacgaccaaaggaccgtcattgaaaccgttaaaatttagcaagagttgggacaagccagtaatcggcaccgtcaaagatactggctattatcttaaatttgcaccagaatctgcagcagagaagtgcacattgcatacgtacaatggtgaatatatcctagatcatttccattatcactgggggaagaaggatggggaaggagcagagcatttcatcgatggaaaacaatacgacatcgagttccactttgtacataaaaaggttgggttgactgatccagatgctagagacgcttttgctgttttgggcgtttttggaaaggccgaccctcgtttgaagatcaatggaatctgggagctactctcaccgtcaactgtcctgactgtcgactcaacacgaaacgtcgctgatgttgttccctctaagcttctcccaagtgccagagactattttcactatgaaggttctttgaccacacctacgtatggtgaggttgtgcactggtttgttctcaatgaacccatagctgtccctagtgagtatctgtcagctctgagacagatgcaagctgacaaagaaggtactgtgattgactcaaactatcgagagcttcaagaagtccacaatcgacctgtgcaacgatttaagagtgatgagcaagggagaggagaatttgacgatatttctaagaatgaggacattgtggaggacttgtctaaattgtctggtaactttattagagagctggtcaggaagatatattggtgacctttttctacacttgttagagttttaggccagaatacatttcatcatttggactgttattttgtgtacactgcttagcagtttatataaacactacaatgccattattataatatagccaatgctgtgatttga

SEQ ID NO: 3 - protein, 330 aa, Suberites domuncula
MLVTVVVLGLLGFASAAQPKFEFVEEWQLWKSTHSKMYESQLMELERHLTWLSNKKYIEQHNVNSHIFGFTLAMNQFGDLSELEYANYLGQYRIEDKKSGNYSKTFQRDPLQDYPEAVDWRTKGAVTAVKDQGDCGASYAFSAMGALEGANALAKGNAVSLSEQNIIDCSIPYGNHGCHGGNMYDAFLYVIANEGVDQDSAYPFVGKQSSCNYNSKYKGTSMSGMVSIKSGSESDLQAAVSNVGPVSVAIDGANSAFRFYYSGVYDSSRCSSSSLNHAMVVTGYGSYNGKKYWLAKNSWGTNWGNSGYVMMARNKYNQCGIATDASYPTL

SEQ ID NO: 4 - DNA, 1200 bp, Suberites domuncula
gaggatagaaagtctacaatctgtaaggacaatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataaacttatatatatatagtcttagaaacattatccttttctttacccttgtctctataggccatagagtgattgtaggctgtttgcatttgatgactgtatataccctatcattttttgtgattctatctgattaaaaatcccatacccgaccaaaccatcaatttatcaaatcatga

SEQ ID NO: 5 - protein, 383 aa, Suberites domuncula
MSALKFVVALCVVHTSLGIAESVGKSKTAGLSDDGNYTAVTKSVRLTPVLEFEEDWKQWTTDHHKVYSDVRERVDKYTVWRANKEYIDQHNQNAQRLGYTLKMNKFGDLTTKEFIEGYHCVQDYQPTNASHLNKKHKTHAFVDYGDFVRGGTGEGVRGVGNMPETMDWRTSGVVTKVKDQLRCGSSYAFSAMASLEGINALSYGSLVTLSEQNIVDCSVTYGNHGCACGDVNRALLYVIENDGVDTWKGYPSGGDPYRSKQYSCKYERQYRGASARGIVSLASGDENTLLTAVANSGPVSVYVDATSTSFQFYSDGVLNVPYCSSSTLSHALVVIGYGKYSGQDYWLVKNSWGPNWGVRGYGKLARNKGNKCGIATAASFPTL

SEQ ID NO: 6 - DNA, 1372 bp, Suberites domuncula
acttagtatattatggtagtgtacaatacctatctccatagaggcatgcataactaggtttattgaatagctgctggcacaatttgttctcaagttggtgctattagatttgtgttctagaatgtcagcattgaagtttgtagttgccttgtgtgtagttcacacaagcttaggaatagctgagtcagttggtaagagcaagactgcaggcctaagtgacgatggcaactacacagctgtcaccaaatctgtaagactgactccagttctagagtttgaggaagattggaagcaatggacaactgatcatcacaaggtctactctgatgtgagggagagagtggacaagtacactgtatggagagctaataaagagtacattgatcaacacaaccagaacgcacagagattgggatacacactcaaaatgaacaaatttggagatttgactaccaaggagttcattgaaggctatcactgtgttcaggactaccaacctaccaatgcatcacatttgaataagaaacacaaaacgcacgcgtttgtcgactatggtgactttgtgaggggtggaactggtgagggtgtgaggggtgtaggaaacatgccggagactatggactggagaacttctggagttgtcacaaaagttaaagatcagcttcgttgtggtagcagctatgcgttctctgccatggcttcattggaaggaataaatgctctttcctacggatctttggtgacactcagtgaacaaaacattgtagactgctcggttacctatggcaaccatggttgcgcctgtggtgatgtaaaccgtgctctactgtatgtgatagagaatgatggcgttgacacgtggaagggttatccttctggtggggatccttatcgatcaaagcaatactcttgcaaatacgagagacagtatcgtggggcctctgctagaggtatagtcagtctagctagtggtgatgagaacacattgttgacagcagtagctaactctggaccagtgagtgtgtatgtggacgctacttcaacatccttccagttttacagtgatggagtgttgaatgttccctattgctcctctagcacgctgagtcatgccttggttgtcattggttacgggaagtacagcggacaagattactggcttgttaaaaacagctggggtcctaactggggagtgcggggctatgggaagttggcaagaaacaagggcaacaaatgtggaatagccacagcggctagtttcccaacattatgacactttagttgatcaaacaattaatcataaattattacaacatgtagtataatgatgcccccccattgctcaatagcttatctttgaacaagaaaaaaa

SEQ ID NO: 7 - protein, 268 aa, Suberites domuncula
MVAISGQAIGPQGPRGLPGRDGRDGQPGSPGSDGQSGSPGRDGRDGLTGPQGVIGEPGPANGPQGPRGLQGTRGEPGSQGPAGQPGPTSGGVVYTRWGKSTCPSVAGTQLVYAGRAGGSEHRDAGAANHLCMPLDPEYTLQHRSGVQGSMYVYGTEYQSPIRGTDNHNVPCAVCSTSTRAQLLMIPAKTSCPTSWTREYYGYLMSQHRSHHPSMYECVDKIKNLSQGVLLTPMVLFSIHVEATCNGMQCPPYNNYKELNCVVCTKRSF

SEQ ID NO: 8 - DNA, 1038 bp, Suberites domuncula
ggcacgaggttaaacagctctcgtgaaaaacatggaaccaaagacctactactctttgctgcagttacactactagtaatgattgtggcaatatcaggtcaggctataggacctcaaggcccacgtggactaccaggaagagatggtcgtgatggacaacctgggtcacctggtagtgatggacaatctggatcacctggtagagatggaagagatggattgactggccctcaaggtgtaataggagaaccaggaccagccaatggaccacaaggacccagaggactacaggggacaaggggagaaccagggtcacaaggacctgctggtcaaccaggacccacgagtggaggtgttgtctacacaaggtgggggaagagcacttgtcctagtgttgctgggactcaactggtatatgctggaagagctggcgggagtgaacacagagatgctggggcagccaaccatctctgtatgcctttagaccctgagtacaccctacaacatcgtagtggagtgcaaggcagtatgtatgtatatggaacagaataccaatctcctattagaggaacagataatcacaacgttccatgtgctgtgtgttcaacatctactcgagcccagctcttaatgattccagctaagacatcctgtccgacatcatggactagagagtactatggctatctcatgtctcaacatcgttcccatcatccttcaatgtatgaatgcgttgacaagatcaagaatctgtcccagggagttctgctaacaccaatggtgctcttttctatccacgttgaagctacctgcaatggtatgcaatgtcctccatacaataactacaaagaactgaactgtgttgtctgcacaaagtaaacttattgacatagtttgtattgcgtgcatgcagtcattaatgtgataggtactgccagtgtgaaaatagagattattgtgggtgtgtgcgtgtgcgtgttttgtgcatgtgtgctgtttttcaatatatcttttattaatataaaaaaaaaaaaaaaa

(19) United States
(12) Patent Application Publication
(10) Pub. No.: US 2008/0293096 A1
(43) Pub. Date: Nov. 27, 2008

(54) Enzyme and Template-Controlled Synthesis of Silica from Non-Organic Silicon Compounds as Well as Aminosilanes and Silazanes and Use Thereof

(76) Inventors: Werner E.G. Muller, Wiesbaden (DE); Heinz C. Schroder, Wiesbaden (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/579,019

(22) PCT Filed: May 2, 2005
(86) PCT No.: PCT/EP2005/004734

§ 371 Date: Jul. 23, 2007

(30) Foreign Application Priority Data
Apr. 30, 2004 (DE) 10 2004 021 230.9

(51) Int. Cl. C12P 1/00

(52) U.S. Cl. 435/41

(57) ABSTRACT

The present invention relates to a method for synthesis of amorphous silicon dioxide (silica, condensation products of silicic acid) and other polymeric metal (IV) compounds from non-organic silicon compounds or metal (IV) compounds as well as from aminosilanes and silazanes, whereby (I) a template (collagen or another molecule, interacting with orthosilicic acid or polymeric silicic acid and salts thereof or other metal (IV) compounds) and (2) a silicase/carbonic anhydrase or a silicatein or similar polypeptide are used for synthesis. Said invention also relates to the technical use thereof.

### 1. STATE OF THE ART

Silicon compounds are extremely significant economically. They are used, among others, in the glass, fiberglass and porcelain industry, in cement production, for producing ceramics, in the paint, rubber, plastic and paper industry, in the detergent industry, in the production of dyes, soaps and cosmetics as well as in medicine/dentistry, e.g., in dental manufacturing/repair. Certain silicates have molecular sieve and ion exchange properties as well as catalytic properties (see, among others: CD Römpp Chemie Lexikon—version 1.0, Stuttgart/New York; Georg Thieme Verlag 1995).

Orthosilicic acid (H₄SiO₄) is a very weak acid. Dilute solutions are only stable for a while at low pH's (pH 2-3). The increasing or decreasing of the pH causes intermolecular splitting off of water (condensation), disilicic acid (pyrosilicic acid; H₆Si₂O₇) occurring as the first condensation product. Other condensation products produced at first—with a rather low link number (n=3, 4 or 6)—are cyclic silicic acids as well as also cage-like silicic acids and polysilicic acids. These metasilicic acids have the gross composition (H₂SiO₃)ₙ. The end product of the condensation is a polymeric silicon dioxide (SiO₂)ₓ that is amorphous, since chain-lengthening and branching processes take place simultaneously in a disordered manner. In all silicic acids, the silicon atoms are present in the center of regular tetrahedrons whose corners each form four oxygen atoms. In the polysilicic acids or in amorphous silicon dioxide, the oxygen atoms simultaneously belong to the adjacent tetrahedrons, which are irregularly linked to each other.

### 1.1 Biosilica

Even the skeleton of siliceous algae (diatoms) and of siliceous sponges consists of amorphous SiO₂ (“biosilica”). The SiO₂ synthesis in these organisms is distinguished by a high (structural) specificity and ability to be regulated, which makes possible the synthesis of defined structures in the microscopic and submicroscopic range (nanostructures). In addition, siliceous sponges have the capacity to form their silicate structures under mild conditions, that is, at a relatively low temperature and a low pressure. This is based on the fact that specific enzymes participate in their synthesis. In contrast thereto, drastic conditions such as high pressure and high temperature are usually necessary for the chemical synthesis of silicates. Therefore, the production of many silicon compounds in traditional manners is cost-intensive and also not very environmentally friendly.

Two enzymes that participate in silicate-forming organisms in the synthesis of the SiO₂ skeleton and their technical use have been described. The first enzyme concerns silicatein, which occurs in three forms, silicatein α, β and γ (PCT/US99/30601. Methods, compositions, and biometric catalysts, such as silicateins and block copolypeptides, used to catalyze and spatially direct the polycondensation of siliconalkoxides, metal alkoxides, and their organic conjugates to make silica, polysiloxanes, polymetallo-oxanes, and mixed poly(silicon/metallo)oxane materials under environmentally benign conditions. Inventors/applicants: D E Morse, G D Stucky, T D Deming, J Cha, K Shimizu, Y Zhou; DE 10037270 A1. Silicatein-vermittelte Synthese von amorphen Silicaten und Siloxanen und ihre Verwendung. German Patent Office 2000. Applicants and inventors: W E G Müller, B Lorenz, A Krasko, H C Schröder; PCT/EP 01/08423. Silicatein-mediated synthesis of amorphous silicates and siloxanes and use thereof. Inventors/applicants: W E G Müller, B Lorenz, A Krasko, H C Schröder). Silicatein α was cloned from the marine siliceous sponge Suberites domuncula (A Krasko, R Batel, H C Schröder, I M Müller, W E G Müller (2000) Expression of silicatein and collágen genes in the marine sponge S. domuncula is controlled by silicate and myotrophin. Europ. J. Biochem. 267:4878-4887). Silicatein β, which was also cloned from S. domuncula, is distinguished by a few advantageous properties in comparison to silicatein α as regards its catalytic capacities and their technical/medical applicability (DE 103 52 433.9. Enzymatische Synthese, Modifikation und Abbau von Silicium(IV)-und anderer Metall(IV)-Verbindungen. German Patent Office 2003. Applicant: Johannes Gutenberg University Mainz; Inventors: W E G Müller, H Schwertner, H C Schröder).

Both silicateins, silicatein α and silicatein β, are only capable according to the state of the art of forming amorphous silicon dioxide (polysilicic acids and polysilicates) from organic silicon compounds (alkoxysilanes) (J N Cha, K Shimizu, Y Zhou, S C Christianssen, B F Chmelka, G D Stucky, D E Morse (1999); Silicatein filaments and subunits from a marine sponge direct the polymerization of silica and silicones in vitro. Proc. Natl. Acad. Sci. USA 96:361-365, as well as the patents cited above).

The second enzyme is a silicase (DE 102 46 186.4. Abbau und Modifizierung von Silicaten und Siliconen durch Silicase und Verwendung des reverisiblen Enzyms. German patent Office 2002. Applicant: Johannes Gutenberg University Mainz. Inventors: W E G Müller, A Krasko, H C Schröder; PCT/EP03/10983. Abbau und Modifizierung von Silicaten und Siliconen durch Silicase und Verwendung des reversiblen Enzyms. European Patent Office 2003. Applicant: Johannes Gutenberg University Mainz. Inventors: W E G Müller, A Krasko, H C Schröder; H C Schröder, A Krasko, G Le Pennec, T Adell, M Wiens, H Hassanein, I M Müller, W E G Müller (2003), Silicase, an enzyme which degrades biogenous amorphous silica: Contribution to the metabolism of silica deposition in the demosponge Suberites domuncula. Prog. Mol. Subcell. Biol. 33:250-268). The silicase, and in particular the enzyme from the marine sponge S. domuncula, is capable of dissolving amorphous as well as crystalline silicon dioxide. This results in the liberation of silicic acid. In addition, the silicase has the ability of dissolving lime material in analogy with carbonic anhydrase.

It was not previously disclosed that this enzyme is also capable in the presence of a suitable template (e.g., collagen) of bringing about a synthesis of amorphous silicon dioxide (silica) from non-organic short-chain metasilicates as well as from aminosilanes or silazanes containing one or several Si—N bonds.

### 1.2. Collagen

Collagen is, in addition to elastin, polyanionic proteoglycans and structural glycoproteins, the primary component of the extracellular matrix of tissues and organs. Collagen fibrils have extraordinarily great tensile strength. As a result, they are especially capable of imparting mechanical stability to the connective and supporting tissue. Furthermore, the formation of collagen fibrils is an important process in wound healing.

In vertebrates the collagens form a large protein family: 19 collagen types have been described that are coded from at least 33 different genes (Prockop and Kivinrikko (1995) Annu. Rev. Biochem. 64:403-434). The members of the collagen family include fibrillary as well as non-fibrillary proteins. The fibrillary collagen types I, II, III, V and XI are capable of forming fibrils with a band pattern. The so-called non-fibrillary collagens occur with the fibrillary collagens (fibril-associated collagens) or in the basal membranes (type IV; basal membrane collagens). Furthermore, the short-chain collagens belong to this group. A few collagens such as types XV and XVIII are known only on the basis of their cDNA.

The common structural feature of all collagens is the triple helix, which consists of three interwoven polypeptide chains (α chains) that have the repeating sequence G-x-y; x is usually proline and y is frequently hydroxyproline. This triplet conditions the characteristic helical conformation of the collagen α helix and its property of assembling with similar polypeptide chains under the formation of the triple helix (Brodska and Ramshaw (1997), Matrix Biol. 15:545-554). The triple helix is usually composed of the polypeptide chains of different collagen types (α1, α2, α3). The resulting structure has great stability on account of the position of glycine (a small amino acid) near the axis of the helix, the stabilizing action of proline and the formation of hydrogen bridge bonds (Bella et al. (1994), Science 266:75-81).

The type I collagen forms the primary amount of the collagen in the organism. The type II collagen is the fibril-forming collagen of the cartilage. In these collagen types three α chains are embedded together. The length of the tropocollagen molecules formed in this manner is 280 nm. An offset arrangement of these components is found in the collagen fibrils. Transverse strips within the collagen fibers occur every 68 nm as a result of the staggered arrangement of these molecules.

In the so-called minority collagens the triple helix is found only in a few sections of the molecule; other sections have globular domains. They include the collagen types IV to XIX. However, the type V and type XI minority collagens also form fibril structures. The type IV collagen is specialized for the formation of spatial checkerworks and occurs in the basal membranes. The type VI collagen occurring in the interstitial connective tissue has only a relatively short triple helix; the two globular domains at the ends of this dumbbell-shaped collagen type interact with the type I collagen as well as with integrins in membrane position. The type VII collagen serves to anchor the basal membrane under squamous epithelia. The type VIII and type X collagens are short-chain collagens; the type VIII collagens associate to a hexagonal network. The type IX collagen belongs to the fibril-associated collagens and occurs together with type II collagen in the calcifying areas of the enchondral cartilage.

1.2.1. Cloning and Sequencing of Collagens from Sponges

Collagen is also a main protein of the extracellular matrix of sponges and functions as matrix for the formation of spicules (formation of sponge needles) (Krasko et al. (2000), Eur. J. Biochem. 267:4878-4887). Collagen fibrils in sponges are very similar to those in vertebrates (Gross et al. (1956), J. Histochem. Cytochem. 4:227-246; Garrone et al. (1975), J. Ultrastruct. Res. 52: 261-275; Garrone (1978) Phylogenesis of connective tissue. Karger, Basel). Electron microscopic examinations of the collagen from the marine sponge Geodia cydonium show 20 to 25 nm thick collagen fibrils with a periodicity of 19.5 nm (Diehl-Seifert et al. (1985), J. Cell Sci. 79:271-285; Gramzow et al. (1988), J. Histochem. Cytochem. 36:205-212). The collagen cloned by us from the marine sponge S. domuncula (Schröder et al. (2000), FASEB J. 14:2022-2031) consists of (i) a non-collagen N-terminal domain, (ii) a collagen internal domain and (iii) a non-collagen C-terminal domain. The internal domain is unusually short in S. domuncula with only 24 G-x-y collagen triplets. In contrast thereto, the collagen of the fresh-water sponge Ephydatia muelleri has two internal domains with 79 G-x-y triplets (Exposito et al. (1991), J. Biol. Chem. 266:21923-21928). The organization of the genes coding for the fibrillary sponge collagen thus greatly resembles that of the vertebrate collagen genes.

The expression of collagen in sponge cells (primmorphs, a special form 3D cell aggregates formed from individual sponge cells were used; DE 19824384. Herstellung von Primmorphe aus dissoziierten Zellen von Schwämmen, Korallen und weiteren Invertebraten: Verfahren zur Kultivierung von Zellen und Schwämmen und weiteren Invertebraten zur Produktion und Detektion von bioaktiven Substanzen, zur Detektion von Umweltgiften und zur Kultivierung dieer Tiere in Aquarien und im Freiland. Inventors and applicants: W E G Müller, F Brümmer; Müller et al. (1999), Mar. Ecol. Prog. Ser. 178:205-219) is stimulated by myotrophin (Schröder et al. (2000) FASEB J. 14:2022-2031; Krasko et al. (2000) Eur. J. Biochem. 267:4878-4887). Myotrophin is a growth-promoting protein that was also cloned by the inventors from S. domuncula.

### 2. SUBJECT MATTER OF THE INVENTION

The inventors were now able to surprisingly show that silicase and other carbonic anhydrases as well as silicateins are capable in the presence of a suitable template such as collagen to also convert non-organic silicon compounds, especially metasilicates, as well as aminosilanes or silazanes containing one or more Si—N bonds into silica. It was previously only known that silicateins catalyze the hydrolysis of organic silicon compounds with one or more Si—O bonds (alkoxysilanes) (with subsequent condensation of the released silanols under formation of amorphous silicon dioxide; see Zhou et al. (1999), Angew. Chem. [int. ed.] 38:780-782; PCT/US99/30601; DE 10037270 A1; PCT/EP01/08423). It was known about the enzymes containing carbonic anhydrase domains that they are capable of splitting inorganic polysilicates (polysilicic acids) as well as amorphous and also crystalline silicon dioxide under the release of silicate acid (Schröder et al. (2003), Prog. Mol. Subcell. Biol. 33:250-268; DE 102 46 108.4; PCT/EP 03/10983) but not, on the other hand, of catalyzing a template-controlled synthesis of amorphous silicon dioxide (silica) from orthosilicates and metasilicates.

Thus, according to a first aspect of the present invention a method for the in vitro or in vivo synthesis of amorphous silicon dioxide (silica, condensation products of silicate acid) and other metal(IV) compounds is made generally available in which a polypeptide or a metal complex of a polypeptide is used that is either characterized in that the polypeptide comprises an animal, vegetable, bacterial or fungal carbonic anhydrase domain exhibiting at least 25%, preferably at least 50%, more preferably at least 75% and most preferably at least 95% sequence identity with the sequence shown in SEQ ID No. 1, or in that the polypeptide comprises an animal, vegetable, bacterial or fungal silicatein α domain or silicatein β domain exhibiting at least 25%, preferably at least 50%, more preferably at least 75% and most preferably at least 95% sequence identity with the sequence shown in SEQ ID No. 3 or in SEQ ID No. 5.

A further aspect of the present invention concerns the use of a template that has a polypeptide of collagen from S. domuncula in accordance with SEQ ID No. 7 or a polypeptide homologous to it that exhibits at least 25%, preferably at least 50%, more preferably at least 75% and most preferably at least 95% sequence identity with the sequence shown in SEQ ID No. 7 in its amino acid sequence or contains parts of it or consists of it.

The template in accordance with the invention (collagen or another polypeptide) can be characterized in that it was synthetically produced or that it is present in a prokaryotic or eukaryotic cell extract or cell lysate. The cell extract or the lysate can be obtained from a cell ex vivo or ex vitro, e.g., from a recombinant bacterial cell or a marine sponge.

The template in accordance with the invention (collagen or another polypeptide) can be purified in accordance with the traditional methods known in the state of the art and thus be present substantially free of other proteins.

A method in accordance with the invention is preferred that is characterized in that compounds such as silicic acids (orthosilicic acid and metasilicic acid) or their salts (orthosilicates and metasilicates) or other metal(IV) compounds are used for the synthesis as reactants (substrates).

Furthermore, a method in accordance with the invention is preferred that is characterized in that compounds such as alkylaminosilanes and dialkylaminosilanes, bis(alkylamino)silanes or bis(dialkylamino)silanes, tris(alkylamino)silanes or tris(dialkylamino)silanes, tetrakis(alkylamino)silanes or (dialkylamino)silanes as well as alkyl-substituted or aryl-substituted derivatives of these compounds (in general: aminosilanes) are used for the synthesis that are characterized in that they contain one or more Si—N bonds.

Furthermore, a method in accordance with the invention is preferred that is characterized in that disilazanes, trisilazanes, tetrasilazanes and polysilazanes as well as alkyl-substituted or aryl-substituted derivatives of these compounds (in general: silazanes), including the cyclic compounds (cyclotrisilazanes, cyclotetrasilazanes and other derivatives) are used for the synthesis.

A further aspect of the present invention is the use of the method for the modification of surfaces of glass, metals, metal oxides, plastics, biopolymers or other materials.

According to another aspect of the present invention the method can be used for the synthesis of defined two-dimensional and three-dimensional structures of amorphous silicon dioxide (silica, condensation products of silicic acid) and other polymeric metal(IV) compounds.

Yet another aspect of the present invention concerns a chemical compound or silica (amorphous silicon dioxide)-containing structure or surface obtained with the method in accordance with the invention.

SEQ ID No. 2 shows the nucleotide sequence of sponge silicase cDNA and SEQ ID No. 1 shows the polypeptide of sponge silicase derived from the nucleotide sequence (SIA_SUBDO). The derived amino acid sequence of sponge silicase has a great similarity with the amino acid sequences of the carbonic anhydrase family. The eukaryotic-type carbonic anhydrase domain (PFAM00194 [www.ncbi.nim.nig.gov]) is found in sponge silicase in the amino acid range of aa₈₇ to aa₃₃₅. Most of the characteristic amino acids that form the eukaryotic-type carbonic anhydrase signature (Fujikawa-Adachi et al. (1999) Biochim. Biophys. Acta 1431:518-524; Okamoto et al. (2001) Biochim. Biophys. Acta 1518:311-316 are also present in sponge silicase.

The carbonic anhydrases form a family of zinc metallic enzymes (Sly and Hu (1995) Annu. Rev. Biochem. 64:375-401). The three zinc-bonding preserved histidine groups are found in the silicase in the amino acids aa₁₈₁, aa₁₈₃ and aa₂₀₆ (see SEQ ID No. 1).

(Partially commercially obtainable) Carbonic anhydrases from other organisms can also be used in addition to sponge silicase in the method according to the invention.

The invention will now be described in more detail in the following with the attached examples but without being limited to them. The attached sequences and figures show:

SEQ ID No. 1: The amino acid sequence of the silicase from S. domucula (SIA_SUBDO) used in accordance with the invention.

SEQ ID No. 2: The nucleic acid sequence of the silicase from S. domuncula used in accordance with the invention.

SEQ ID No. 3: The amino acid sequence of the silicatein α from S. domuncula (SIA_SUBDO) used in accordance with the invention.

SEQ ID No. 4: The nucleic acid sequence of the silicatein α from S. domuncula used in accordance with the invention.

SEQ ID No. 5: The amino acid sequence of the silicatein β from S. domuncula (SIA_SUBDO) used in accordance with the invention.

SEQ ID No. 6: The nucleic acid sequence of the silicatase β from S. domuncula used in accordance with the invention.

SEQ ID No. 7: The amino acid sequence of the collagen 3 from S. domuncula (SIA_SUBDO) used in accordance with the invention.

SEQ ID No. 8: The nucleic acid sequence of the collagen 3 from S. domuncula used in accordance with the invention.

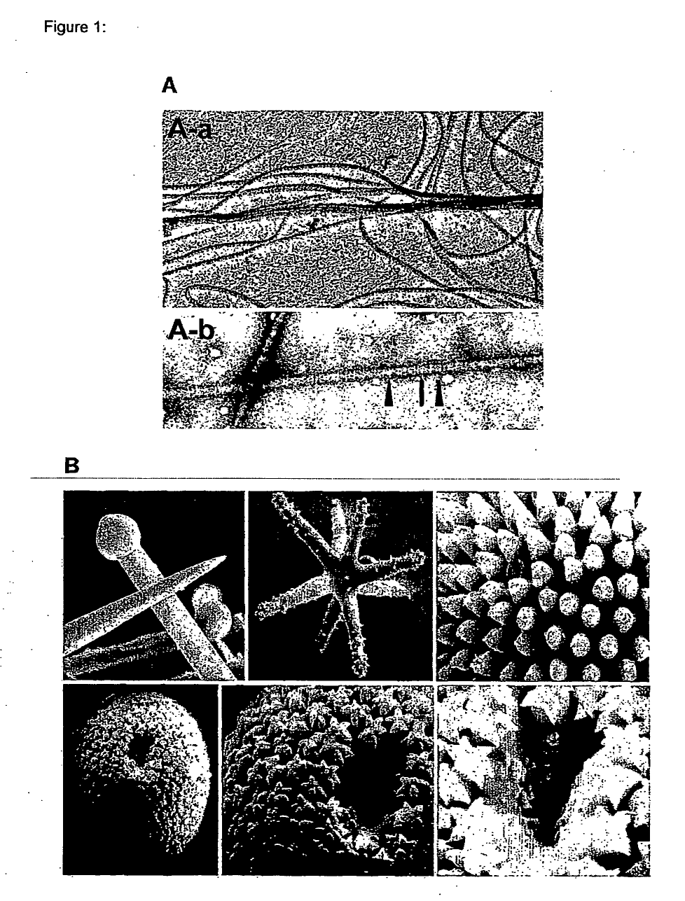

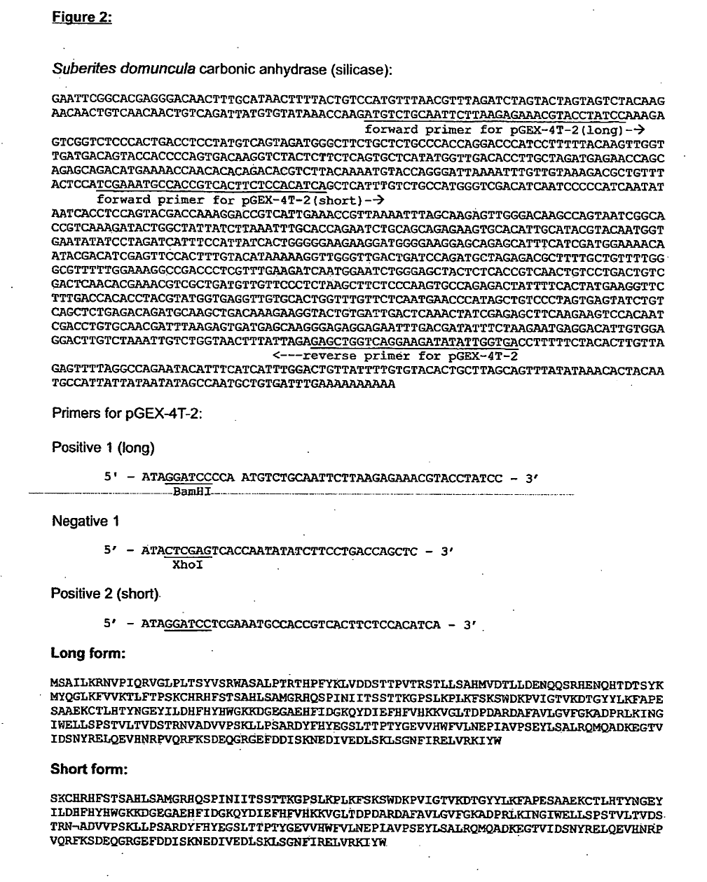

FIG. 1:

A. Electron microscope photographs of isolated collagen from Geodia cydonium. (A-a) bundles of collagen fibrils. (A-b) Negatively colored fibrils. B. Scanning electron microscope photographs of sponge SiO₂ skeleton elements. Top from left to right: Tylostyle (Suberites domuncula), spheraster (Geodia cydonium), sterraster (Geodia cydonium). Bottom from left to right Sterraster (Geodia cydonium) in increasing magnification.

FIG. 2:

Nucleotide sequence of the carbonic anhydrase (silicase) clone (S. domuncula) as well as forward primer (positive 1 and positive 2) and reverse primer (negative 1) for amplifying the cDNA coding for the long and the short silicase form for cloning into the expression vector pGEX-4T-2 and amino acid sequence of the recombinant proteins (long and short form of silicase). Protein information about the proteins is:

Protein information about CAexpresL.prt (long form): Molecular weight: 43130.74 daltons+25000 DaGST˜˜>68 kDa
379 amino acids 46 Strongly basic (+) amino acids (K,R) 46 Strongly acidic (−)amino acids (D,E)120 hydrophobic amino acids (A,I,L,F,W,V)103 polar amino acids (N,C,Q,S,T,Y)7.666 isoelectric point 2.871 charge at pH 7.0
Protein information about CAexpresS.PRO(1, 2-4) (short form: Molecular weight: 32271.28 daltons+25000 daltons GST˜˜>57 kDa284 amino acids 35 Strongly basic (+)amino acids (K,R) 39 Strongly acidic (−)amino acids (D,E)91 hydrophobic amino acids (A,I,L,F,W,V)70 polar amino acids (N,C,Q,S,T,Y)6.701 isoelectric point −1.795 charge at pH 7.0

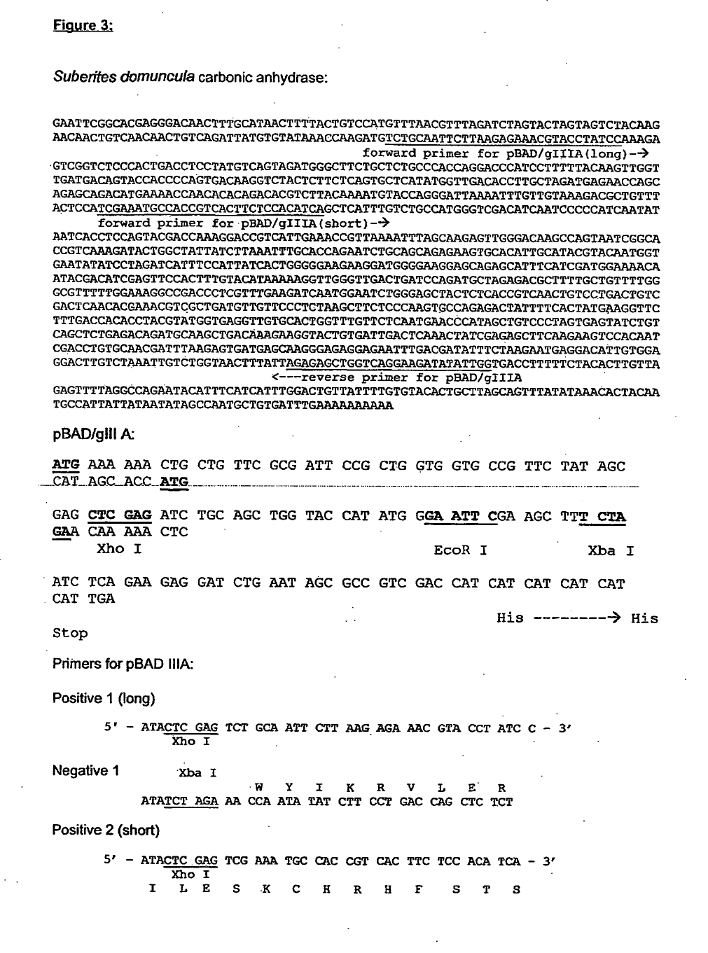

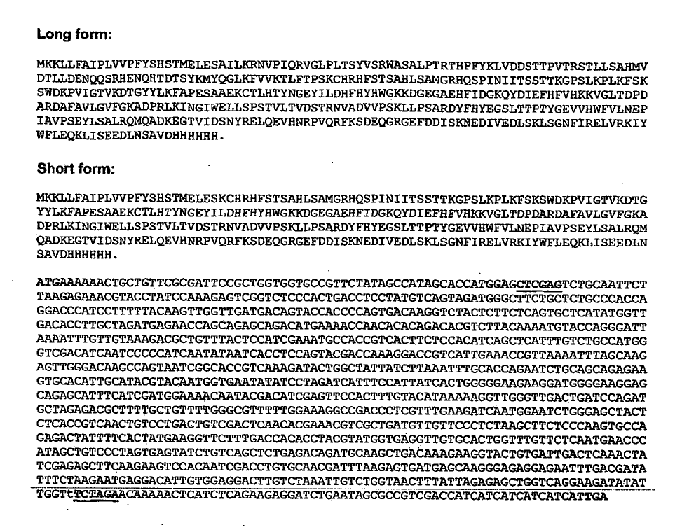

FIG. 3:

Nucleotide sequence of the carbonic anhydrase (silicase) clone (S. domuncula) as well as forward primer (positive 1 and positive 2) and reverse primer (negative 1) for amplifying the cDNA coding for the long and the short silicase form for cloning into the expression vector pBAD/gIII and amino acid sequence of the recombinant proteins (long and short form of silicase). Protein information about the proteins is:

Long form: Molecular weight: 48430.78 daltons
424 amino acids
49 Strongly basic (+) amino acids (K,R)
53 Strongly acidic (−) amino acids (D,E)
137 hydrophobic amino acids (A,I,L,F,W,V)
111 polar amino acids (N,C,Q,S,T,Y)
7.005 isoelectric point
0.045 charge at pH 7.0
Short form: Molecular weight: 33702.52 daltons
330 amino acids
0.045 charge at pH 6.52

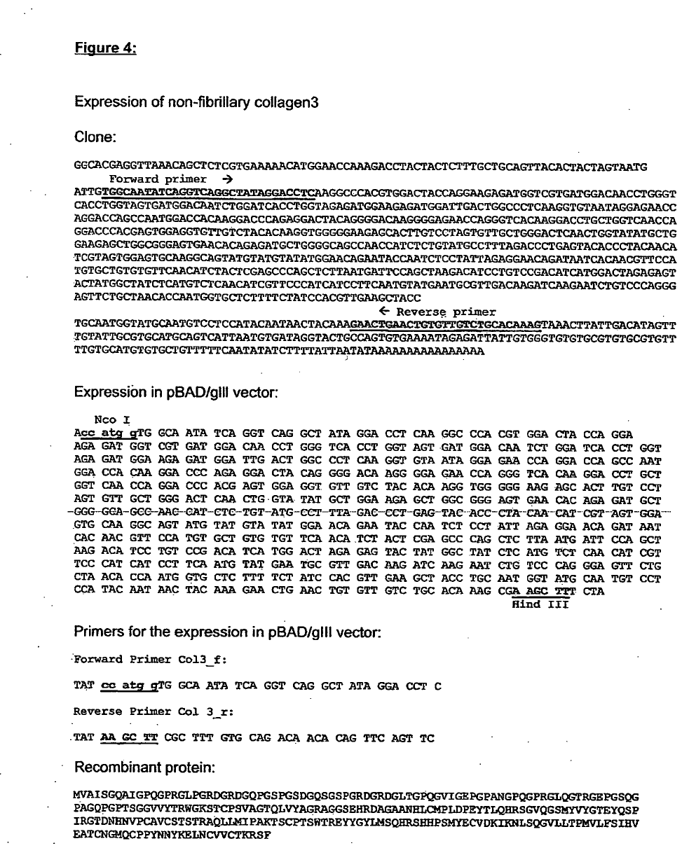

FIG. 4:

Expression of non-fibrillary collagen 3 from S. domuncula in the pBAD/gIII expression vector. The following are shown from top to bottom: Nucleotide sequence of the collagen 3 clone with bonding sites of the forward primer and of the reverse primer; inserted sequence of the non-fibrillary collagen 3 from S. domuncula in the expression vector pBAD/gIII (the restriction sites of NcoI and HindIII are underlined); the primers used for the expression in pBAD/gII (forward primer Col3_f and reverse primer Col_r; the restriction sites of NcoI and HindIII are marked); amino acid sequence of the recombinant protein derived from the nucleotide sequence.

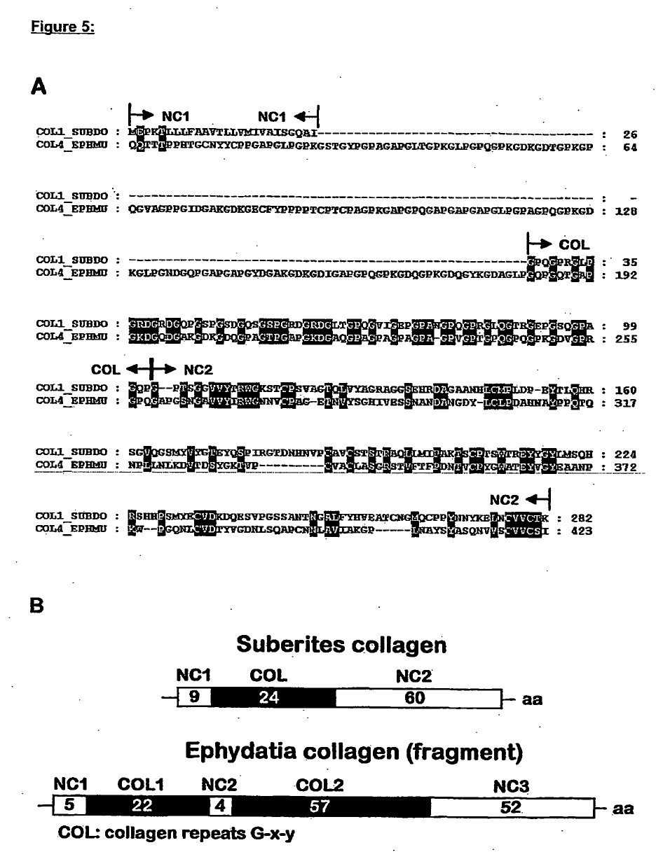

FIG. 5:

Sponge collagens. A. Comparison of the deduced amino acid sequence of the cDNA of S. domuncula collagen (COL1_SUBDO) with those of the collagen from E. muelleri (COL4_EPHMU). Preserved amino acid groups (similar or related as regards their physico-chemical properties) in the sequence are shown in white on black. NC1: Non-collagenic N-terminal domain. COL: Collagenic internal domain. NC2: Non-collagenic C-terminal domain. B. Comparison of S. domuncula collagen with the collagen from E. muelleri. NC1: Non-collagenic C-terminal domain. COL: Collagenic internal domain. NC2: Non-collagenic C-terminal domain. Numbers: Number of amino acids.

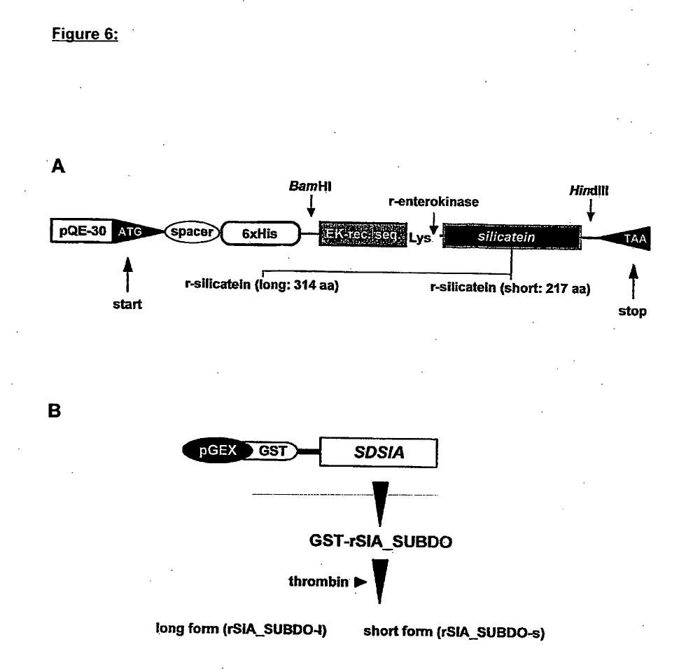

FIG. 6:

A. Production of recombinant silicatein α. B. Production of recombinant silicase.

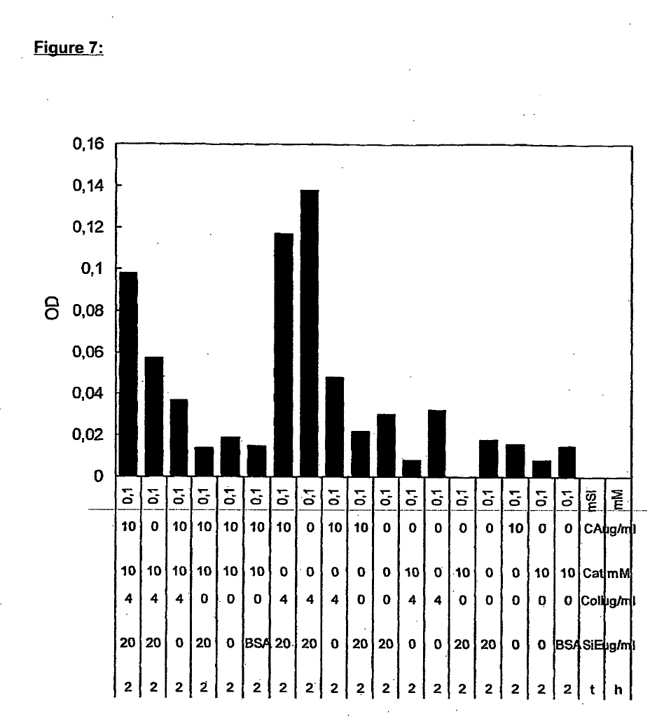

FIG. 7:

In the experiment shown here 100 μM Na metasilicate was incubated in the absence or presence of 20 μg/ml recombinant silicatein α or bovine serum albumin (BSA) in buffer (50 mM tris-HCl pH 7.0, 100 mM NaCl, 0.1 mM ZnSO₄ and 0.1 mM β mercaptoethanol) for 10 min at room temperature. Then, as indicated in the figure, 4 μg/ml recombinant sponge collagen, 10 μg/ml carbonic anhydrase (from bovine erythrocytes) and/or 10 mM catachol were added and incubated for another 2 h at room temperature. All indicated concentrations are the end concentration after the addition of all components to the batches. In order to demonstrate the amorphous silicon dioxide formed, the reaction batches were centrifuged in a table centrifuge (10,000×g; 15 min; 4° C.), washed with ethanol and air-dried. The sediments were subsequently hydrolyzed with 1 M NaOH and the released silicate quantitatively measured using a molybdate-supported demonstration method (colorimetric silicon test of the Merck company).

The test shows that maximal amounts of amorphous silica are synthesized in the presence of collagen, silicatein α and carbonic anhydrase (0.098-0.117 OD units) as well as in the presence of collagen and silicatein α (0.138 OD units). Lesser amounts of non-soluble SiO₂ were determined in the absence of carbonic anhydrase (0.057 OD units) and in the absence of silicatein α (0.037 and 0.048 OD units). In the absence of collagen only very small amounts of non-soluble SiO₂ (0.014-0.019 or 0.022 or 0-0.018 or 0.008 OD units) were measured both with as well as without silicatein or carbonic anhydrase or both enzymes. Likewise, even in the presence of collagen alone only a little non-soluble SiO₂ was formed (0.008 and 0.032 OD units). In the presence of BSA instead of silicatein and collagen only very small amounts of SiO₂ were measured (0.015 OD units) both with as well as without carbonic anhydrase. The addition of catachol resulted in a decrease of the amount of non-soluble SiO₂.

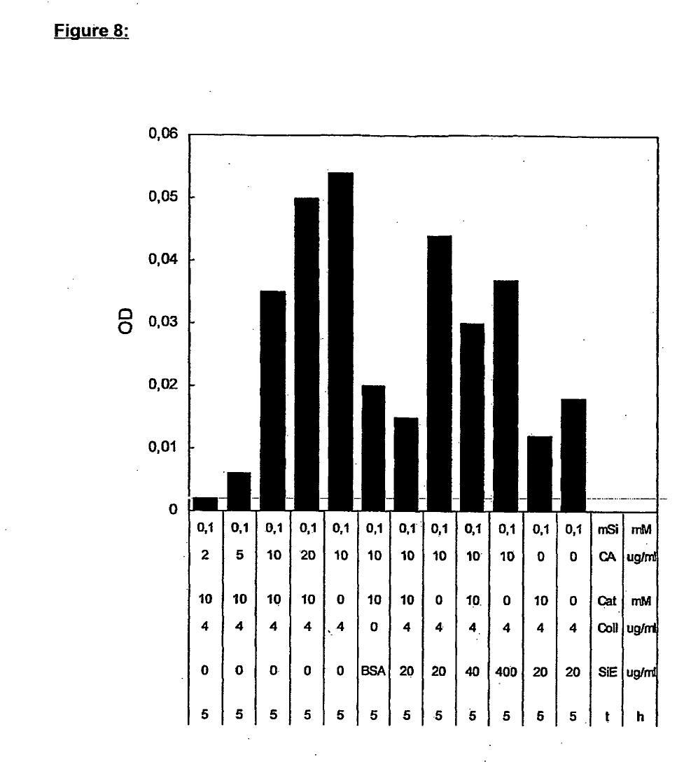

FIG. 8:

In the experiment shown here 100 μM Na metasilicate was incubated in the absence or presence of 20 to 400 μg/ml recombinant silicatein α or bovine serum albumin (BSA; 20 μg/ml) in buffer (50 mM tris-HCl pH 7.0, 100 mM NaCl, 0.1 mM ZnSO₄ and 0.1 mM β mercaptoethanol) for 10 min at room temperature. Then, as indicated in the figure, 4 μg/ml recombinant sponge collagen, 10 μg/ml carbonic anhydrase (bovine erythrocytes) and/or 10 mM catachol were added and incubated for another 5 h at room temperature. All indicated concentrations are the end concentration after the addition of all components to the batches. In order to demonstrate the amorphous silicon dioxide formed, the reaction batches were treated further as described in FIG. 5 and the amount of non-soluble SiO₂ formed was determined. It was found that the amount of non-soluble SiO₂ rises with an increasing concentration of carbonic anhydrase (from 0.002 to 0.050 OD units). A pre-incubation with silicatein α (10 min) did not result in a further increase but rather under the conditions used in a reduction in the formation of SiO₂ (0.015 and 0.030). In the presence of BSA instead of silicatein and collagen only very slight amounts of SiO₂ were measured (0.020 OD units). Without the addition of catachol the amounts of non-soluble SiO₂ formed were greater.

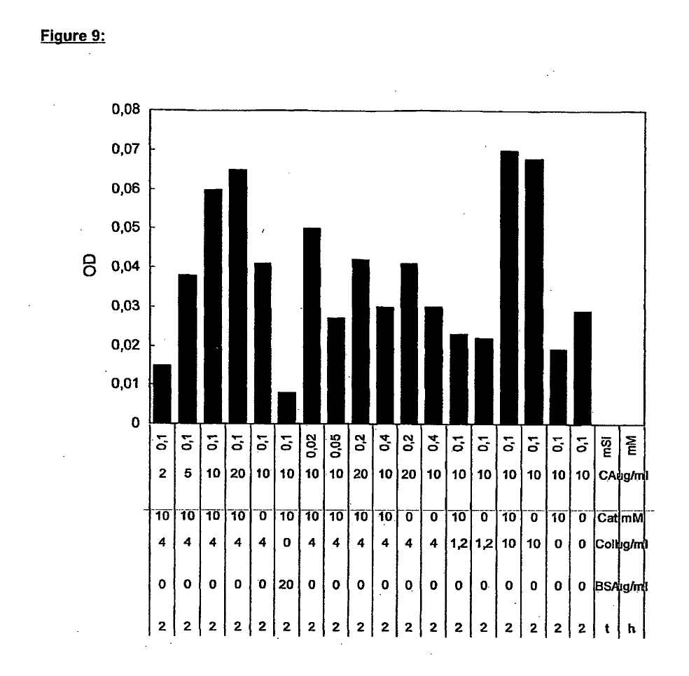

FIG. 9:

In the experiment shown here 100 μM Na metasilicate and 4 μg/ml recombinant sponge collagen were incubated in the presence of rising concentrations (2 to 20 μg/ml) of carbonic anhydrase (from bovine erythrocytes) in buffer (50 mM tris-HCl pH 7.0, 100 mM NaCl, 0.1 mM ZnSO₄ and 0.1 mM β mercaptoethanol) in the presence of 10 mM catechol for 2 h at room temperature. The amount of non-soluble SiO₂ formed rose sharply (from 0.015 to 0.060 OD units). Likewise, the amount of SiO₂ formed rose sharply with an increasing amount of collagen (1.2 to 10 μg/ml) (from 0.022-0.023 to 0.068-0.070 OD units). An increase of the Na metasilicate concentration did not result in a further rise but rather in a reduction of the formation of SiO₂ (up to 0.027 OD units). In the presence of bovine serum albumin (BSA; 20 μg//ml) instead of collagen only very little SiO₂ was formed (0.008 OD units); on the other hand, in the presence of carbonic anhydrase alone the formation of SiO₂ was approximately 0.019-0.029 OD units. Without the addition of catechol the formation of SiO₂ was somewhat less. The indicated concentrations were the end concentration after the addition of all components to the batches. In order to demonstrate the amorphous silicon dioxide formed the reaction batches were treated further as described in FIG. 5 and the amount of non-dissolved SiO₂ formed was determined.

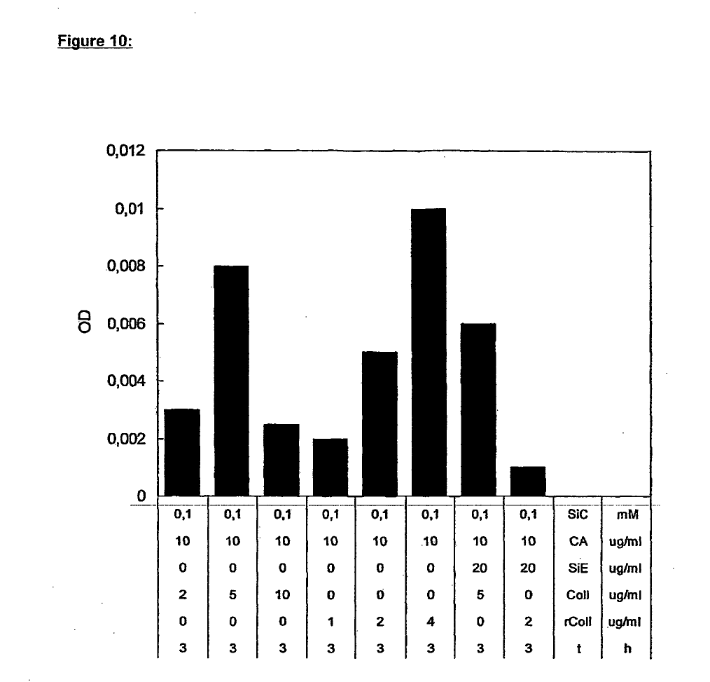

FIG. 10:

In the experiment shown here 100 μM Si-catecholate complex was incubated in the absence or presence of 20 μg/ml recombinant silicatein α in buffer (50 mM tris-HCl pH 7.0, 100 mM NaCl, 0.1 mM ZnSO₄ and 0.1 mM β mercaptoethanol) for 10 min at room temperature. Then, as indicated in the figure, either recombinant sponge collagen (1 to 4 μg/ml) or purified bovine collagen (2 to 10 μg/ml) as well as 10 μg/ml carbonic anhydrase (from bovine erythrocytes) was added and incubated for another 3 h at room temperature. The indicated concentrations were the end concentration after the addition of all components to the batches. In order to demonstrate the amorphous silicon dioxide formed the reaction batches were treated further as described in FIG. 5 and the amount of non-dissolved SiO₂ formed was determined. The results show that upon the addition of increasing amounts of fibrillary collagen (bovine)—in contrast to recombinant, non-fibrillary sponge collagen—the amount of non-soluble SiO₂ formed rises at first but then drops again. Just as in the use of Na metasilicate (see FIG. 7), the amount of SiO₂ formed rose sharply with an increasing amount of sponge collagen (1 to 4 μg/ml) (from 0.002 to 0.010 OD units). In a manner similar to the one in the results obtained with Na metasilicate (see FIG. 5) the formation of SiO₂ was less in the presence of catechol, which can be explained by a shift of the equilibrium in the direction of the Si-catecholate complex. No non-soluble SiO₂ was formed in the presence of carbonic anhydrase alone (not shown in the illustration). An increase in the concentration of recombinant silicatein α to 40 and 400 μg/ml resulted in a reduction of the formation of SiO₂ (not shown in the illustration).

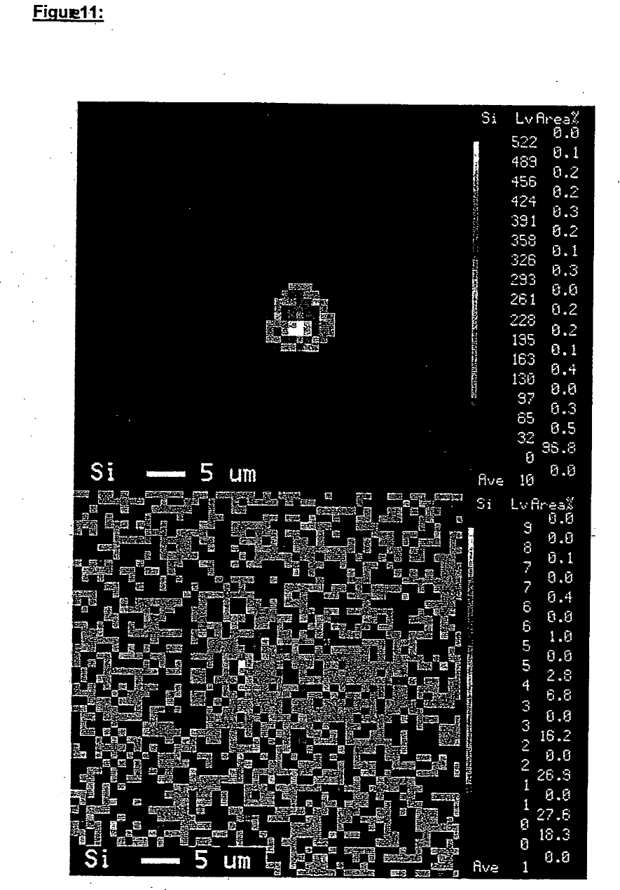

FIG. 11:

The demonstration of the silica products formed is shown with the aid of a High Performance Field Emission Electron Probe Microanalyzer (EPMA). The incubation was carried out in the absence (=control) or in the presence of 50 μg/ml carbonic anhydrase (from bovine erythrocytes; Calbiochem company) and 30 μg/ml collagen in buffer (50 mM tris-HCl pH 7.0, 100 mM NaCl, 0.1 mM ZnSO₄ and 0.1 mM β mercaptoethanol) with 1 mM Na metasilicate at room temperature. The incubation time was 4 h. The results of the elementary analysis for Si in a batch with carbonic anhydrase and collagen (A) and of a control (absence of carbonic anhydrase and collagen; B) are shown.

### 3. PRODUCTION AND DEMONSTRATION OF THE COMPONENTS REQUIRED FOR THE METHOD

### 3.1. Production of Silicase

The purification of silicase from natural sources such as tissues or cells as well as the recombinant production of the enzyme have been described and are state of the art (DE 102 46 186.4. Abbau und Modifizierung von Silicaten und Siliconen durch Silicase und Verwendung des reversiblen Enzyms. German patent Office 2002. Applicant: Johannes Gutenberg, University Mainz. Inventors: W E G Müller, A Krasko, H C Schröder; PCT/EP03/10983. Abbau und Modifizierung von Silicaten und Siliconen durch Silicase und Verwendung des reversiblen Enzyms. European Patent Office 2003. Applicant: Johannes Gutenberg University Mainz. Inventors: W E G Müller, A Krasko, H C Schröder).

The cDNA (SDSIA) coding for the silicase from the marine sponge S. domuncula as well as the polypeptide (SIA_SUBDO) derived from the nucleotide sequence have the following properties. Length of the cDNA: 1395 nucleotides (nt); open reading frame: from nt₁₂₂-nt₁₂₄ to nt₁₂₅₉-nt₁₂₆₁ (stop codon); length of the polypeptide: 379 amino acids; relative molecular mass (Mᵣ) of the polypeptide: 43131; isoelectric point (pI): 6.5.

The recombinant S. domuncula silicase was produced as glutathione S transferase (GST) fusion protein for the experiments described here. A long as well as a shortened fragment of the cDNA (called SDSIA) coding for S. domuncula silicase were cloned into a pGEX-4T-2 plasmid that contained the GST gene (FIG. 2) The results for the purified short form of the silicase with a size of 32 kDa are shown in the following; analogous results are obtained for the long form (Mᵣ 43 kDa), that is, however, less efficient.

Another alternative is the production of recombinant silicase in E. coli using the oligo-histidine expression vector pBAD/gIIIA (Invitrogen), in which the recombinant protein is secreted into the periplasmatic space on account of the gene III signal sequence (FIG. 3). The cDNA sequence coding for silicase (short form) is amplified with PCR using the following primers: Forward primer: ATACTC GAG TCG AAA TGC CAC CGT CAC TTC TCC ACA TCA and reverse primer: ATATCT AGA AA CCA ATA TAT CTT CCT GAC CAG CTC TCT; and cloned into pBAD/gIIIA (restriction nucleases for insertion into the expression vector: XhoI and XbaI). After the transformation of E. Coli XL1-blue the expression of the fusion protein is induced with L-arabinose.

Likewise, an insert can also be used that comprises the entire derived silicase protein (long form).

### 3.2. Production of Silicatein

The purification of silicase α and silicatein β from natural sources such as tissues or cells as well as the recombinant production of the enzymes have been described and are state of the art (DE 10037270 A 1. Silicatein-vermittelte Snythese von amorphen Silicaten und Siloxanen und ihre Verwendung. German Patent Office 2000. Applicants and inventors: W E G Müller, B Lorenz, A Krasko, H C Schröder; PCT/EP 01/08423. Silicatein-mediated synthesis of amorphous silicates and siloxanes and use thereof. Inventors/applicants: W E G Müller, B Lorenz, A Krasko, H C Schröder; DE 103 52 433.9. Enzymatische Synthese, Modifikation und Abbau von Silicium(IV)-und anderer Metall(IV)-Verbindungen. German Patent Office 2003. Applicant: Johannes Gutenberg University Mainz; Inventors: W E G Müller, H Schwertner, H C Schröder).

The production of the recombinant silicatein α in E. coli took place for the experiments described here using the oligo-histidine expression vector pBAD/gIIIA (Invitrogen), in which the recombinant protein is secreted into the periplasmatic space on account of the gene III signal sequence. The cDNA sequence coding for silicase (short form) is amplified with PCR using the following primers: Forward primer: TAT CC ATG GAC TAC CCT GM GCT GTA GAC TGG AGA ACC and reverse primer TAT T CTA GA A TTA TAG GGT GGG ATA AGA TGC ATC GGT AGC; and cloned into pBAD/gIIIA (restriction nucleases for insertion into the expression vector: NcoI and XbaI). After the transformation of E. Coli XL1-blue the expression of the fusion protein is induced with L-arabinose.

The recombinant sponge-silicatein polypeptide (short form) has a molecular weight of ˜28.5 kDa (˜26 kDa silicatein plus 2 kDa vector) and an isoelectric point of pI 6.16.

Likewise, an insert can also be used that comprises the entire derived silicatein α protein (long form).

### 3.3. Production of Sponge Collagen

Both native collagen (from vertebrates such as, e.g., bovine collagen as well as from invertebrates (such as, e.g., from marine demosponges)) as well as recombinant collagen (especially from the marine sponge S. domuncula) can be used as template. A few methods for their preparation are described in the following.

### 3.3.1. Isolation of Native Sponge Collagen

A simple method for isolating collagen from various marine sponges has been described (DE 100 10 113 A 1. Verfahren zur Isolierung von Schwammkollagen sowie Herstllung von nanopartikulärem Kollagen. Applicant: W. Schatton. Inventors: J Kreuter, W E G Müller, W. Schatton, D Swatschek, M Schatton; Swatschek et al. (2002) Eur. J. Pharm. Biopharm. 53:107-113). The sponge collagen is obtained with a high yield (<30%).

### 3.3.2. Production of Recombinant Sponge Collagen

The clone used to produce the recombinant collagen codes for a non-fibrillary collagen (collagen 3) from the marine sponge Suberites Domuncula; this collagen has the advantage that it (1) has a relatively low molecular weight and (2) is not modified further posttranslationally.

The cDNA sequence coding for collagen 3 can be amplified with PCR using suitable primers and subcloned into a suitable expression vector. The expression was carried out successfully with, among others, the oligo-histidine expression vectors pBAD/gIIIA (Invitrogen) and pQTK_{—}1 (Qiagen). The following can be used as primers for the PCR (with the following use of pBAD/gIIIA): Forward primer: TAT cc atg gTG GCA ATA TCA GGT CAG GCT ATA GGA CCT C and reverse primer: TAT AA GC TT CGC TTT GTG CAG ACA ACA CAG TTC AGT TC; restriction nucleases for insertion into the expression vector: NcoI and HindIII. After transformation of Escherichia coli strain XL1-blue with the plasmid (expression vector) the expression of the fusion protein is induced with L-arabinose (at pBAD/gIIIA) or with isopropyl-β-D-thiogalactopyranoside (IPTG; at pQTK_{—}1). The expression vector pBAD/gIIIA has the advantage that the recombinant protein is secreted into the periplasmatic space on account of the gene III signal sequence. The signal sequence is removed after the membrane passage. When pQTK_{—}1 is used the bacteria are extracted with PBS/8 M urea. The suspension is centrifuged after ultrasonic treatment. The purification of the fusion protein from the supernatant takes place by metal-chelate affinity chromatography using an Ni-NTA agarose matrix (Qiagen) as described by Hochuli et al. (J. Chromatogr. 411:177-184; 1987). The extract is placed on the column, subsequently washed with PBS/urea and the fusion protein eluted from the column with 150 mM imidazol in PBS/urea.

The characterization of the collagen preparations takes place via SDS-PAGE, determination of the amino acid composition, of the isoelectric point as well as by electron microscopy.

Molecular weight, isoelectric point. The determination of the molecular weights can take place by SDS-PAGE. The molecular weight of the protein obtained after expression of the cDNA amplified using the above-cited primers is ˜28.5 kDa.

The isoelectric point (IEP) can be determined by titration in aqueous solution. The IEP of sponge collagen is mostly approximately pH 6.5-8.5 (for comparison, IEP of bovine collagen: pH 7.0±0.09). The peptide (see SEQ ID No. 7) derived from the cDNA shown in SEQ ID No. 8 has a previously stated isoelectric point of 8.185. The charge at pH 7.0 is 4.946.

Amino acid composition: The determination of the amino acid composition can be carried out with the aid of an automatic amino acid analyzer.

Electron microscopy. The electron microscopic characterization of the isolated sponge collagen can take place by transmission electron microscopy (TEM). To this end the freeze-dried collagen sample is negatively contrasted with a 2% phosphorus-tungsten acid (Harris, Negative staining and cryoelectron microscopy. Royal Microscopical Society Microscopy Handbook No. 35. BIOS Scientific Publishers Ltd., Oxford, UK).

### 3.4. Demonstration of Silicase Activity

The method for the demonstration of silicase activity of (commercial) carbonic anhydrase preparations (e.g., from bovine erythrocytes; Calbiochem company) and/or of recombinant sponge silicase has been described (DE 102 46 186.4; PCT/EP03/10983).

### 3.5. Demonstration of Silicatein Activity

The method for the demonstration of silicatein activity (silicatein α and silicatein β) has been described (PCT/US99/30601; DE 10037270 A 1; PCT/EP01/08423; DE 103 52 433.9).

The silicic acid can be quantitatively determined, e.g., with the aid of a molybdate-supported demonstration method such as, e.g., the calorimetric silicon test (Merck; 1.14794). The amount of silicic acid can be calculated using a calibration curve for the silicon standard (Merck 1.09947) from the extinction values at 810 nm.

### 4. DESCRIPTION OF THE METHOD OF SILICA SYNTHESIS

In the method in accordance with the invention, silicic acid is incubated, with a template and an enzyme, in the form of a metasilicate (sodium salt or salt of another alkali, alkaline earth or metal ion), silicon complex (that is in equilibrium with free orthosilicic acid or orthosilicate; e.g., silicon catecholate [dipotassiumtricatecholateosilicon]) or in the form of orthosilicic acid or of an orthosilicate in a suitable buffer (e.g., 50 mM tris-HCl pH 7.0, 100 mM NaCl, 0.1 mM ZnSO₄ and 0.1 mM β mercaptoethanol or other buffers; the presence of Zn is advantageous in the incubation with silicase or carbonic anhydrases, that constitute Zn enzymes) for a period adapted to the desired amount of the silica product formed (amorphous silicon dioxide). The incubation can be carried out at different temperatures. Room temperature (22° C.) has proved to be advantageous but higher (e.g., 37° C.) or lower temperatures (e.g., 15° C.) have also been used successfully.

To this end, the metasilicate can either be dissolved in the buffer used or previously (possibly as a rather highly concentrated stock solution) in an alkaline solution (such as 0.01 N NaOH). In the latter instance, the metasilicate solution obtained must be neutralized (pH: 7.2 more advantageous).

The template is one or several different molecules, molecular aggregates or surfaces comprising functional groups that interact with orthosilicic acid, oligomeric or polymeric silicic acids as well as their salts (orthosilicates, metasilicates).

It proved to be advantageous if the molecules containing hydroxyl groups are collagen or a silicatein (see FIG. 7-10).

It proved to be especially advantageous if the collagen is a collagen from a sponge, in particular a collagen according to SEQ ID No. 7 or a polypeptide homologous to it that exhibits at least 25%, preferably at least 50%, more preferably at least 75% and most preferably at least 95% sequence identity in its amino acid sequence with the sequence shown in SEQ ID No. 7 or parts of it. The collagen indicated in SEQ ID No. 7 is a non-fibrillary collagen (collagen 3) from the marine sponge S. domuncula. This collagen proved to be more efficient than fibrillary bovine collagen (see FIG. 10).

Furthermore, it proved to be especially advantageous if the silicatein is a silicatein from a sponge in accordance with SEQ ID No. 3 or a polypeptide homologous with it that exhibits at least 25%, preferably at least 50%, more preferably at least 75% and most preferably at least 95% sequence identity in its amino acid sequence with the sequence shown in SEQ ID No. 3 or parts of it (see FIGS. 7-10).

Aside from silicatein α (SEQ ID No. 3), silicatein β (SEQ ID No. 5) or a polypeptide homologous with it that exhibits at least 25%, preferably at least 50%, more preferably at least 75% and most preferably at least 95% sequence identity in its amino acid sequence with the sequence shown in SEQ ID No. 5 or parts of it can also be used.

A mixture of one or more templates (e.g., collagen and silicatein) can also be used (see FIGS. 7-10).

The collagen from a sponge in accordance with SEQ ID No. 7 or a polypeptide homologous with it that exhibits at least 25%, preferably at least 50%, more preferably at least 75% and most preferably at least 95% sequence identity in its amino acid sequence with the sequence shown in SEQ ID No. 7 or parts of it can be made available in vivo, in a cell extract or cell lysate or in purified form.

The enzyme is a polypeptide of a silicase from Suberites domuncula in accordance with SEQ ID No. 1 or a polypeptide homologous with it that exhibits at least 25%, preferably at least 50%, more preferably at least 75% and most preferably at least 95% sequence identity in the amino acid sequence of the carbonic anhydrase domain with the sequence shown in SEQ ID No. 1, a metal complex of the polypeptide or parts of it (see FIGS. 7-10).

The polypeptide of a silicase from S. domuncula in accordance with SEQ ID No. 1 or a polypeptide homologous with it that exhibits at least 25%, preferably at least 50%, more preferably at least 75% and most preferably at least 95% sequence identity in the amino acid sequence of the carbonic anhydrase domain with the sequence shown in SEQ ID No. 1 can be made available in vivo, in a cell extract or cell lysate or in purified form.

The use of commercial carbonic anhydrases such as the carbonic anhydrase from bovine erythrocytes is also advantageous (see FIGS. 7-10).

The addition of catechol, that complexes free silicic acid, results in a decrease of the amount of non-soluble SiO₂ (see FIGS. 7 and 10).

Maximal amounts of amorphous silica are synthesized in the presence of collagen, silicatein and carbonic anhydrase as well as in the presence of collagen and silicatein (see FIG. 7). Lesser amounts of non-soluble SiO₂ are obtained in the presence of collagen and carbonic anhydrase (see FIG. 7). A pre-incubation with silicatein can also result in a reduction in the formation of SiO₂ (see FIG. 8) depending on the conditions applied (incubation time). In the absence of collagen only very slight amounts of non-soluble SiO₂ are formed with silicatein or carbonic anhydrase or with both enzymes (see FIG. 7). Control experiments with BSA instead of silicatein and collagen as template show only a very slight formation of non-soluble SiO₂ (see FIGS. 7-9).

The amount of non-soluble SiO₂ formed rises with an increasing concentration of carbonic anhydrase (see FIGS. 8 and 9).

Furthermore, the amount of SiO₂ formed is a function of the concentration of the template used; a rise is found with a rising concentration, e.g., of silicatein α (see FIG. 8) or of collagen (see FIG. 9).

An increase in the concentration of Na metasilicates did not result in a further increase but rather in a reduction of the formation of SiO₂ (see FIG. 9).

Aside from metasilicates silicon complexes (e.g., the silicon-catechol complex) can also be used; here too the amount of SiO₂ formed rises with an increasing amount of collagen (see FIG. 10). However, when the silicon-catechol complex is used instead of metasilicates the yields of non-soluble SiO₂ are less (cf. FIGS. 7-9 and FIG. 10).

The use of other silicon complexes such as the silicon complexes with gallic acid or tropolone (tristropolonatosilicon chloride) is also possible.

The incubation with silicatein and carbonic anhydrase can be carried out simultaneously (see FIG. 9) or successively (see FIGS. 7, 8 and 10).

Aside from collagen a number of other biomaterials and composite materials can serve as template for the formation of silica such as fibrillary chitin obtained in accordance with a described method (DE 102 10 571.5. Zusammensetzung und Verfahren zur Herstellung von modifiziertes fibrilläres Chitin und poenzierende Zusatzstoffe enthaltenden, biologisch hochaktiven Präparaten und ihre Anwendung als Protektions-und Nahrungsergänzungsmittel wäthrend der prä-und postnatalen Entwicklung und adulter Lebensphasen bei Mensch und Tier. Applicants and inventors: W E G Müller, H C Schröder, B Lorenz, O F Senyuk, L F Gorowoj).

The method is also suitable for the synthesis of other polymeric metal(IV) compounds from purely inorganic metal(IV) compounds wherein (1) a template (molecule, molecular aggregate or surface) and (2) a polypeptide or a metal complex of a polypeptide are also used for the synthesis, that is either characterized in that the polypeptide comprises an animal, vegetable, bacterial or fungal carbonic anhydrase domain exhibiting at least 25%, sequence similarity with the sequence shown in SEQ ID No. 1, or in that the polypeptide comprises an animal, vegetable, bacterial or fungal silicatein α domain or silicatein 8 domain exhibiting at least 25%, preferably at least 50%, more preferably at least 75% and most preferably at least 95% sequence identity with the sequence shown in SEQ ID No. 3 or in SEQ ID No. 5.

4.1. Demonstration of the Silicon Dioxide formed

In order to demonstrate the products (amorphous silicon dioxide formed), the material (or the reaction batch) can be centrifuged in a table centrifuge (12,000×g; 15 min; 4° C.), washed with ethanol and air-dried. The sediment can be subsequently hydrolyzed with 1 M NaOH. The released silicate is quantitatively measured in the produced solution using a molybdate-supported demonstration method such as, e.g., the colorimetric silicon test of the Merck company.

The demonstration of the silica product formed (element analysis) can also take place with the aid of a High Performance Field Emission Electron Probe Microanalyzer (EPMA). A JXA-8900RL Electron Probe Microanalyzer (JEOL, Inc, Peabody, Mass., USA) was used for the experiment shown in FIG. 11. This apparatus combines high-resolution scanning electron microscopy (REM) with high-quality x-ray analysis.

The batches for the analysis with the High Performance Field Emission Electron Probe Microanalyzer contained 1 mM Na metasilicates in 50 mM tris-HCl pH 7.0, 100 mM NaCl, 0.1 Mm ZnSO₄ and 0.1 mM β mercaptoethanol. The incubation was carried out in the absence (=controls) or in the presence of 50 μg/ml carbonic anhydrase (from bovine erythrocytes; Calbiochem company) and 30 μg/ml collagen for 4 h at room temperature.

100 μl of the samples (batches after incubation) were placed onto each of the carriers. The carriers with the preparations were subjected to a carbon vapor-deposition (Emitech K959) under a vacuum (10⁻⁴ mbar). Ca, Na and Cl were determined in addition to Si.

The results showed that a distinct formation of silicon aggregates was able to be demonstrated in the batches with carbonic anhydrase and collagen but not, on the other hand, in the controls (absence of carbonic anhydrase and collagen) (see FIG. 11).

No conformity resulted in the localizations of the signals for Si, Ca, Na and Cl.

### 5. USES OF THE METHOD

A number of different industrial and technical uses result for the described method for the enzymatic synthesis of amorphous silica from inorganic (non-organic) silicon compounds, namely:

1.) The use for the surface modification of biomaterials that consist either of the cited template materials (molecules containing hydroxyl groups) themselves or coated with them. This can also be surfaces of glass, metals, metal oxides, plastics, biopolymers or other materials. An overview of literature concerning surface-modified biomaterials is found in: B D Ratner et al. (editors) Biomaterials Science—An Introduction to Materials in Medicine. Academic Press, San Diego, 1996. The conditions used in traditional physical/chemical methods for producing these modifications often have a detrimental (destructive) effect on the biomaterials. The method in accordance with the invention uses, in comparison to the traditional methods, “mild” conditions that are gentle on the biomaterials since it is based solely on biochemical/enzymatic reactions. In particular, a use for the method in accordance with the invention also results in the production of surface modifications (coating) of collagen that serves as replacement material for tissue, bone, or teeth, and of collagen fleeces (tissue engineering). The surface modifications serve to increase the stability and the porosity as well as to improve the ability to resorb.

The advantages of sponge collagen as biomaterial are, as with other collagens, biodegradability as well as a low toxicity and immunogenicity. However, sponge collagen does not have the disadvantages of the collagen that was previously primarily obtained from animal skins and the bones of swine, calves and cattle in which the possibility of an infection by pathogenic germs cannot be excluded.

A further advantage of the method is the fact that no organic solvents have to be used to dissolve the initial substrate used (silicic acids and metasilicates as well as their salts), as is the case with organic silicon compounds (e.g., TEOS). This avoids damage to the biopolymers to be modified as well as to collagen.

2.) The use for the modification or the synthesis of nanostructures of silica (amorphous silicon dioxide). It is possible with the method in accordance with the invention to synthesize defined two-dimensional and three-dimensional structures of silica (or of other polymeric metal(IV) compounds) on a nanoscale from purely inorganic initial substrates (silicic acid, metasilicic acid and their salts). The structures formed can be used in nanotechnology.

3.) The use of the method in accordance with the invention to produce three-dimensional silica-coated matrices of collagens with defined physical and chemical properties for producing tissues/organs of the human organism with autologous body cells that can be used as replacement tissue for treating oncological defects, posttraumatic organ and tissue damage, burn injuries, vascular occlusions as well as surgical wounds. The special advantage of the method in accordance with the invention is that (1) reactions of incompatibility and of rejection by the receiving organism are avoided by the silica coating and (2) no damage to the matrices (collagen) by organic solvents can occur (the initial substrates are water-soluble in contrast to the organic silicon compounds such as TEOS to be used according to the state of the art).

1. A method for the synthesis of amorphous silicon dioxide and other polymeric metal(IV) compounds wherein (1) a template with (2) non-organic silicon compounds or metal(IV) compounds and/or aminosilanes and silazanes as substrate and (3) with a polypeptide or a metal complex of a polypeptide are brought in contact for synthesis, wherein the polypeptide comprises an animal, vegetable, bacterial or fungal carbonic anhydrase domain exhibiting at least 25% sequence identity with the sequence shown in SEQ ID No. 1, or the polypeptide comprises an animal, vegetable, bacterial or fungal silicatein α domain or silicatein β domain exhibiting at least 25% sequence identity with the sequence shown in SEQ ID No. 3 or in SEQ ID No. 5.

2. The method according to claim 1, characterized in that a template is used for the synthesis that comprises functional groups that interact with orthosilicic acid, oligomeric or polymeric silicic acids as well as their salts or with other purely non-organic metal(IV) compounds or aminosilanes or silazanes.

3. The method according to claim 1, characterized in that compounds such as orthosilicic acid, oligomeric or polymeric silicic acids as well as their salts or other metal(IV) compounds are used as substrate.

4. The method according to claim 1, characterized in that aminosilanes or silazanes containing one or several Si—N bonds are used as substrate for the synthesis.

5. The method according to claim 1, wherein the templates are molecules, molecular aggregates or surfaces containing hydroxyl groups.

6. The method according to claim 5, wherein the molecules containing hydroxyl groups are collagen or silicatein or both.

7. The method according to claim 6, wherein the collagen is a collagen from a sponge.

8. The method according to claim 7, wherein the collagen is a collagen from a sponge in accordance with SEQ ID No. 7 or a polypeptide homologous to it that exhibits at least 25% sequence identity in its amino acid sequence with the sequence shown in SEQ ID No. 7 or parts of it.

9. The method according to claim 6, wherein the silicatein is a silicatein from a sponge in accordance with SEQ ID No. 3 or a polypeptide homologous to it that exhibits at least 25% sequence identity in its amino acid sequence with the sequence shown in SEQ ID No. 3 or SEQ ID No. 5 or parts of it.

10. The method according to claim 1, wherein a mixture of one or several templates is used.

11. The method according to claim 1, wherein a polypeptide of a silicase from Suberites domuncula in accordance with SEQ ID No. 1 or a polypeptide homologous to it that exhibits at least 25% sequence identity with the sequence shown in SEQ ID No. 1 in the amino acid sequence of the carbonic anhydrase domain, a metal complex of the polypeptide or parts of it is/are used.

12. The method according to claim 1, wherein the polypeptide of a silicase from Suberites domuncula according to SEQ ID No. 1 or a polypeptide homologous to it that exhibits at least 25% sequence identity with the sequence shown in SEQ ID No. 1 in the amino acid sequence of the carbonic anhydrase domain is made available in vivo, in a cell extract or cell lysate or in purified form.

13. The method according to claim 7, wherein the collagen from a sponge in accordance with SEQ ID No. 7 or a polypeptide homologous to it that exhibits at least 25% sequence identity with the sequence shown in SEQ ID No. 7 or parts of it in its amino acid sequence is made available in vivo, in a cell extract or cell lysate or in purified form.

14. The method according to claim 1, wherein glass, metals, metal oxides, plastics, biopolymers or other materials are modified as surfaces.

15. The method according to claim 1, wherein defined two-dimensional and three-dimensional structures of amorphous silicon dioxide or other polymeric metal(IV) compounds are synthesized.
